(19) United States
(12) Patent Application Publication
(10) Pub. No.: US 2010/0056931 A1
(43) Pub. Date: Mar. 4, 2010

(54) CARDIAC OUTPUT ESTIMATION USING PULMONARY ARTERY PRESSURE

(76) Inventors: Leah Soffer, Minneapolis, MN (US); Haresh G. Sachanandani, Culver City, CA (US); Bin Mi, Plymouth, MN (US); Yunlong Zhang, Mounds View, MN (US)

Correspondence Address: SCHWEGMAN, LUNDBERG & WOESSNER/BSC-CRM, PO BOX 2938, MINNEAPOLIS, MN, 55402, US

(21) Appl. No.: 12/543,227

(22) Filed: Aug. 18, 2009

Related U.S. Application Data
(60) Provisional application No. 61/091,863, filed on Aug. 26, 2008.

(51) Int. Cl. A61B 5/0215; A61B 5/029

(52) U.S. Cl. 600/486; 600/526

(57) ABSTRACT

A system and method sense a pressure signal in a pulmonary artery and compute a stroke volume and cardiac output. A pressure signal is received from an implantable pressure sensor disposed in a pulmonary artery. The pressure signal includes a systolic period and a diastolic period for determining a heart rate (HR) and a heart cycle. An iteratively-updating model can relate pressure signal and HR to a stroke volume (SV) and a cardiac output (CO). The model extracts a mean pulse pressure (MPP) from the PAP signal and receives a patient-specific vascular resistance model parameter and a patient-specific arterial compliance model parameter. CO can be calculated using the HR, the PAP signal, and the model. The vascular resistance model parameter and the arterial compliance model parameter are iteratively updated using the output of the model.

### CROSS-REFERENCE TO RELATED APPLICATION

This application claims the benefit of U.S. Provisional Application No. 61/091,863, filed on Aug. 26, 2008, under 35 U.S.C. §119(e), which is hereby incorporated by reference.

### BACKGROUND

The heart is the center of the circulatory system of the human body. The left-sided chambers of the heart, including the left atrium and the left ventricle, draw blood from the lungs and pump it to the organs of the body to provide the organs with oxygenated blood. The right-sided chambers of the heart, including the right atrium and the right ventricle, draw blood from the organs and pump it into the lungs where the blood gets oxygenated. The organs of the body require oxygen to survive, which requires adequate cardiac output and adequate blood flow. Stroke volume and cardiac output are hemodynamic parameters that can be used to determine or characterize a patient's heart status.

### Overview

The present inventors have recognized, among other things, that inability to easily determine stroke volume or cardiac output can impact the therapy provided to cardiac patients. With the large number of patients receiving implantable cardiac rhythm management devices (e.g., pacers, defibrillators, resynchronization devices, etc.), it would be economically and clinically beneficial to be able to more accurately estimate stroke volume or cardiac output on a chronic basis to help manage patients and improve outcomes.

Approaches to measuring cardiac output using thermo-dilution or the Fick method are invasive techniques that necessitate the patient to be present in a clinical or hospital setting. Other non-invasive techniques using Doppler ultrasound also require use in hospital or clinic settings due to the expense of equipment and need for trained technicians.

Stroke volume and cardiac output may be calculated from flow measurements by integrating the flow to determine stroke volume. One approach uses differential pressure measurement to estimate flow, but this method requires pressure measurements to be taken from two different locations. However, it is preferable to use just a single pressure measurement to reduce system complexity, especially when measurements are from implantable devices. Wireless sensors implanted in the heart and great vessels provide many advantages for monitoring pressure, including direct measurements of clinically valuable data, such as pressure in pulmonary artery. Additionally, placing a sensor within the right side of the vasculature minimizes risks associated with the left side, including blood clots and potential for stroke. This type of sensor system is desirable because it can lead to providing various new types of heart failure therapy using the sensor signal information. Moreover, several implantable sensors may also be used in conjunction with each other to further determine patient health status.

This document discusses, among other things, a system and method for estimating stroke volume and cardiac output using a model as described herein that receives a pressure signal from a single Pulmonary Artery Pressure (PAP) sensor. The estimated stroke volume and cardiac output of the heart can be detected using a PAP signal, or a parameter derived from the signal, using a change in the PAP, using an interval between multiple PAP signal features, or using information from the PAP and information from a different physiological signal, including an electrical cardiac signal, a heart sound signal, posture sensor signal, and/or an oxygen saturation signal.

In Example 1, an apparatus comprises an input receiving a pressure sensor signal from an implantable pressure sensor; an output providing an output signal used to calculate a cardiac output; a memory, configured to store a patient-specific vascular resistance model parameter and a patient-specific arterial compliance model parameter; and a processor coupled to the input, the output and the memory, wherein the processor is configured to: receive the pressure sensor signal, the pressure sensor signal including a systolic period and a diastolic period; determine a heart rate (HR) of a heart cycle; provide an iteratively-updating model that relates the pressure sensor signal and HR to at least one of a stroke volume (SV) and a cardiac output (CO), and that extracts a measure of pulse pressure derived from the pressure sensor signal, the model using a patient-specific vascular resistance model parameter and a patient-specific arterial compliance model parameter; calculate the CO using the HR, the pressure sensor signal, and the model; iteratively update the patient-specific vascular resistance model parameter using the output signal from the model; and iteratively update the patient-specific arterial compliance model parameter using the output signal from the model.

In Example 2, the apparatus of Example 1, wherein the implantable pressure sensor optionally comprises a pulmonary artery pressure (PAP) sensor disposed in a pulmonary artery of a patient.

In Example 3, the apparatus of Example 1-2, optionally comprises a processor configured to receive a patient-specific vascular impedance model parameter stored in the memory.

In Example 4, the apparatus of Examples 1-3 optionally comprises a processor configured to determine a pulmonary blood flow profile over the heart cycle using the pressure sensor signal, the vascular resistance model parameter and the arterial compliance model parameter that are stored in the memory.

In Example 5, the apparatus of Examples 1-4 optionally comprises a processor configured to determine the stroke volume by integrating the pulmonary blood flow profile over the heart cycle.

In Example 6, the apparatus of Examples 1-5 optionally comprises a processor configured to determine a second arterial compliance model parameter using the pressure sensor signal.

In Example 7, the apparatus of Example 6 optionally comprises a processor configured to determine the second arterial compliance model parameter using the stroke volume and a pulse pressure extracted from the pressure sensor signal.

In Example 8, the apparatus of Examples 1-7 optionally comprises a processor configured to determine a second vascular resistance model parameter using the pressure sensor signal.

In Example 9, the apparatus of Example 8 optionally comprises a processor configured to determine a second vascular resistance model parameter using a central tendency of the pressure sensor signal and a central tendency of the pulmonary blood flow profile over the heart cycle.

In Example 10, the apparatus of Example 9 optionally comprises a processor configured to replace the vascular resistance model parameter with the second vascular resistance model parameter.

In Example 11, the apparatus of Example 10 optionally comprises a processor configured to replace the arterial compliance model parameter with the second arterial compliance model parameter.

In Example 12, the apparatus of Example 1-11 optionally configured to filter and down-sample the pressure sensor signal to generate the pulmonary blood flow profile.

In Example 13, the apparatus of Examples 1-12 optionally comprises a processor configured to determine the heart rate and the heart cycle by identifying the systolic period and the diastolic period in the pressure sensor signal.

In Example 14, the apparatus of Examples 1-13 optionally comprises a processor configured to identify the systolic period and the diastolic period by identifying a dicroctic notch in the pressure sensor signal during the heart cycle.

In Example 15, the apparatus of Example 14 optionally comprises a processor configured to identify the dicrotic notch using peak detection.

In Example 16, the apparatus of Example 14 optionally comprises a processor configured to identify the dicrotic notch using a physiological signal generated from a second physiological sensor.

In Example 17, the apparatus of Example 16 optionally comprises additional physiological sensor, which may include at least one of a heart sound sensor or ECG monitor.

In Example 18, the apparatus of Examples 1-17 optionally comprises a processor configured to determine a central tendency of the CO over a specified number of heart cycles.

In Example 19, the apparatus of Example 1-18 optionally comprises a posture sensor to normalize cardiac output calculations.

In Example 20, the apparatus of Examples 1-19 optionally comprises attenuating a respiration effect of the patient from the calculated CO.

In Example 21, the apparatus of Example 1-20 optionally comprises an implantable medical device configured to receive the pressure sensor signal from the PAP sensor.

In Example 22, the apparatus of Example 21 optionally comprises a processor disposed in the implantable device capable of computing the cardiac output.

In Example 23, the apparatus of Example 21 optionally comprises a processor disposed in the PAP sensor capable of computing the cardiac output.

In Example 24, the apparatus of Examples 1-23 optionally comprises an external device in communication with the implantable device and configured to receive the cardiac output signal from a processor disposed in the implantable device.

In Example 25, the apparatus of Example 1-24 optionally comprises an external device configured to receive the pressure sensor signal from the PAP sensor.

In Example 26, the apparatus of Example 25 optionally comprises a processor disposed in the external device capable of computing the cardiac output.

In Example 27, a method comprises receiving a pulmonary artery pressure (PAP) signal from an implantable pressure sensor disposed in a pulmonary artery of a patient, the PAP signal including a systolic period and a diastolic period; determining a heart rate (HR) of a heat cycle; providing an iteratively-updating model that relates PAP and HR to at least one of a stroke volume (SV) and a cardiac output (CO), and that extracts a measure of pulse pressure (MPP) from the PAP, the model using a patient-specific vascular resistance model parameter and a patient-specific arterial compliance model parameter; calculating the CO using the HR, the PAP signal, and the model; iteratively updating the patient-specific vascular resistance model parameter using the outputs of the iteratively updating model; and iteratively updating the patient-specific arterial compliance model parameter using the output of the iteratively updating model.

In Example 28, the method of Example 27 optionally comprises determining a pulmonary blood flow profile over the heart cycle using the PAP signal, the vascular resistance model parameter and the arterial compliance model parameter.

In Example 29, the method of Examples of 27-28 optionally comprises determining the stroke volume by integrating the pulmonary blood flow profile over the heart cycle.

In Example 30, the method of Examples 27-29 optionally comprises determining the heart rate by identifying the systolic period and the diastolic period in the pressure waveform.

In Example 31, the method of Example 30 optionally comprises identifying the systolic period and the diastolic period by identifying a dicroctic notch in the PAP signal during the heart cycle using secondary physiological sensors.

In Example 32, the method of Example 30 optionally comprises identifying the systolic period and the diastolic period using peak detection.

In Example 33, the method of Examples 27-32 optionally comprises determining a pulmonary blood flow profile over the heart cycle using the patient-specific vascular resistance model parameter, the patient-specific arterial compliance model parameter, and the pulmonary artery pressure (PAP) signal; determining a stroke volume and a pulse pressure; generating an updated arterial compliance parameter using the stroke volume and the pulse pressure; determining a central tendency of the PAP signal and a central tendency of the pulmonary blood flow profile over the heart cycle; generating an updated vascular resistance parameter using the central tendency of the PAP signal and the central tendency of the pulmonary blood flow profile; replacing the patient-specific vascular resistance model parameter with the updated vascular resistance parameter; and replacing the patient-specific arterial compliance model parameter with the updated arterial compliance parameter.

In Example 34, the method of Examples 27-33 optionally comprises filtering and down-sampling the PAP signal before determining the pulmonary blood flow profile.

In Example 35, the method of Examples 27-34 optionally comprises determining a central tendency of the updated vascular resistance parameter and the updated arterial compliance parameter over a specified number of heart cycles.

In Example 36, the method of Examples 27 35 optionally comprises determining a central tendency of the cardiac output over a specified number of heart cycles.

In Example 37, the method of Examples 27-36 optionally comprises providing the iteratively updating model in an implantable medical device capable of being communicatively coupled to the implantable pressure sensor.

In Example 38, the method of Examples 27-36 optionally comprises providing the iteratively updating model in an external device capable of being communicatively coupled to the implantable pressure sensor.

In Example 39, the method of Examples 27-38 optionally comprises receiving at least one baseline signal from a plurality of physiological sensors including a posture sensor and adjusting the cardiac output using the baseline signal.

In Example 40, a system comprises means for receiving a pulmonary artery pressure (PAP) signal from a pulmonary artery pressure sensor disposed in a pulmonary artery of a patient, the PAP signal including a systolic period and a diastolic period; means for determining a heart rate (HR) of a heat cycle; means for providing an iteratively-updating model that relates PAP and HR to at least one of a stroke volume (SV) and a cardiac output (CO), and that extracts a measure of pulse pressure (MPP) from the PAP, the model using a patient-specific vascular resistance model parameter and a patient-specific arterial compliance model parameter; means for calculating the CO using the HR, the PAP signal, and the model; means for iteratively updating the patient-specific vascular resistance model parameter using the model outputs; and means for iteratively updating the patient-specific arterial compliance model parameter using the model outputs.

In Example 41, the system of Example 40 optionally comprises means for receiving a pulmonary artery pressure by an implantable device.

In Example 42, the system of Example 40 optionally comprises means for receiving a pulmonary artery pressure by an external device.

This overview is intended to provide a summary of the subject matter of the present patent application. It is not intended to provide an exclusive or exhaustive explanation of the invention. The detailed description is included to provide further information about the subject matter of the present patent application.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 illustrates an example of a two-element electrical analogue model of a vascular system used to relate blood flow to pressure waveform using two lumped parameters.

FIG. 2 illustrates an example of a three-element electrical analogue model of a vascular system used to relate blood flow to pressure waveform using three lumped parameters.

FIG. 3 illustrates an example of a system including an implantable pulmonary artery pressure (PAP) sensor.

FIG. 4 illustrates generally an example of a processing system configured to receive a pulmonary artery pressure signal, stored patient specific parameters, such as resistance, compliance, impedance, and generate values for stroke volume and cardiac output.

FIG. 5 is an example system showing a detailed view of the processing system in FIG. 4 to generate a cardiac output signal.

FIG. 6 illustrates generally an example of a system including a PA pressure sensor, a processor, and an auxiliary physiological sensor.

FIG. 7 illustrates an example of a method for estimating the stroke volume and cardiac output of a patient.

### DETAILED DESCRIPTION

In heart failure patients, the heart's ability to perform work is impaired. The ability of the heart to eject blood in each beat (Stroke Volume, SV) and the liters of blood pumped by the left ventricle per minute (Cardiac Output, CO) are significantly reduced. An ejection fraction (EF) of 20% or less is very common with the New York Heart Association (NYHA) class III/IV heart failure patients. Measuring stroke volume and cardiac output allows physicians to evaluate a patient's heart performance.

This document describes, among other things, a real time CO monitoring system with inputs from an implantable sensor that allow for ongoing, daily monitoring of stroke volume, cardiac output, and heart function, in general. Additionally, the systems and methods provided herein allows for remotely monitoring the cardiac output and stroke volume of a patient.

The present approach can incorporate a pulse contour method (PCM), which has been developed from the “Windkessel” theory, which provides a method of deriving cardiac output and stroke volume from an arterial pressure pulse wave by modeling the vascular system using similar relations as an electrical circuit.

The method can relate a blood flow to a pressure waveform at a specific physiological location (such as an aortic artery), by using a set of lumped parameters such as arterial compliance, vascular peripheral resistance, vascular impedance.

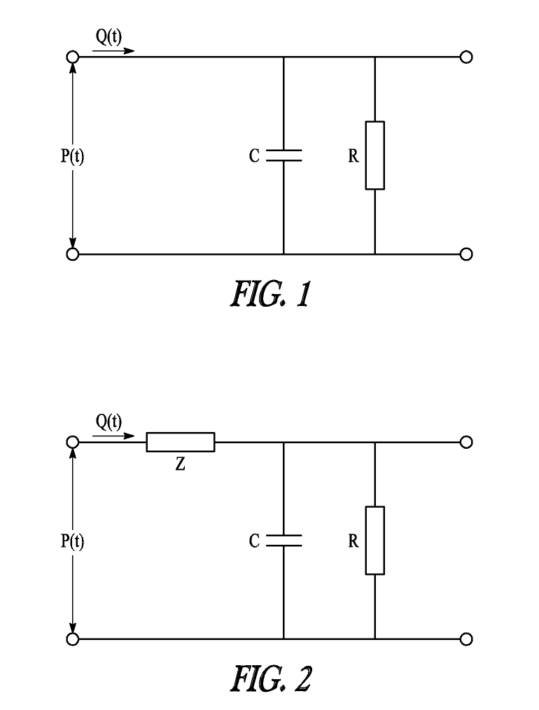

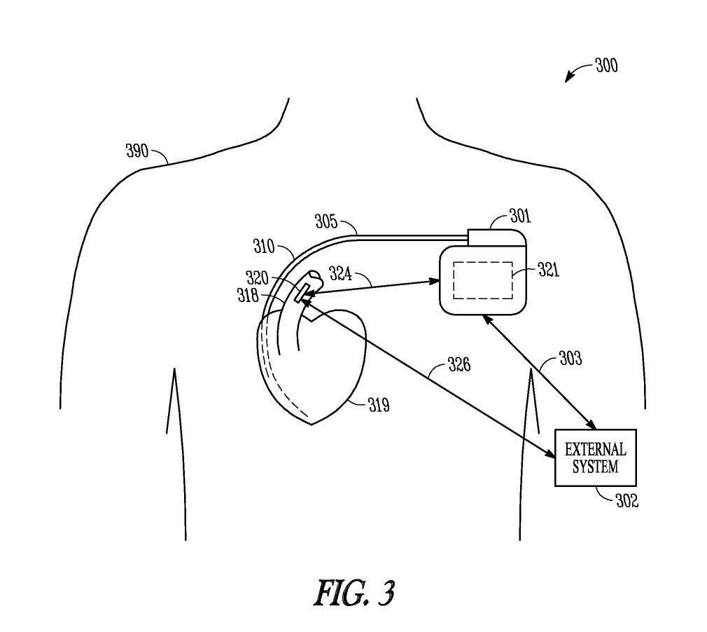

Table 1 shown below lists the various electrical analogues of hemodynamic equivalents shown in FIGS. 1 and 2.

TABLE 1

| Circuit Element | Hemodynamic Property |
|---|---|
| Current | Blood flow (Q) |
| Potential | Blood pressure (P) |
| Capacitance | Arterial compliance (C) |
| Resistance | Vascular peripheral resistance (R) |
| Impedance | Vascular impedance (Z) |

The equivalent time domain equations derived from the circuits shown in FIG. 1 and FIG. 2 are provided below as Equation 1 and Equation 2, respectively.

FIG. 1 illustrates a two-element electrical analogue model of a vascular system used to relate blood flow to pressure waveform using two lumped parameters. The two-element electrical circuit shown in FIG. 1 includes capacitance C and resistance R that represent arterial compliance and vascular peripheral resistance, respectively.

The two element time domain relationship between various vascular parameters is provided as follows:

$$Q = C \frac{d P}{d t} + \frac{P}{R} \qquad ( Equation 1 )$$

Wherein C=compliance and R resistance

FIG. 2 illustrates a three-element electrical analogue model of a vascular system used to relate blood flow to pressure waveform using three lumped parameters. The three-element model has three principal components, which include: the impedance which represents the opposition of the blood vessel to pulsatile flow or systemic resistance, the arterial compliance which represents the opposition of the blood vessel to volume increases, and the vascular peripheral resistance which represents the opposition of the vascular structure to blood flow.

The three element time domain relationship between various vascular parameters is provided as follows:

$$\frac{d P}{d t} = \frac{R + Z}{RC} Q + Z \frac{d Q}{d t} - \frac{P}{RC} \qquad ( Equation 2 )$$

wherein, C=compliance, R=resistance and Z=impedance.

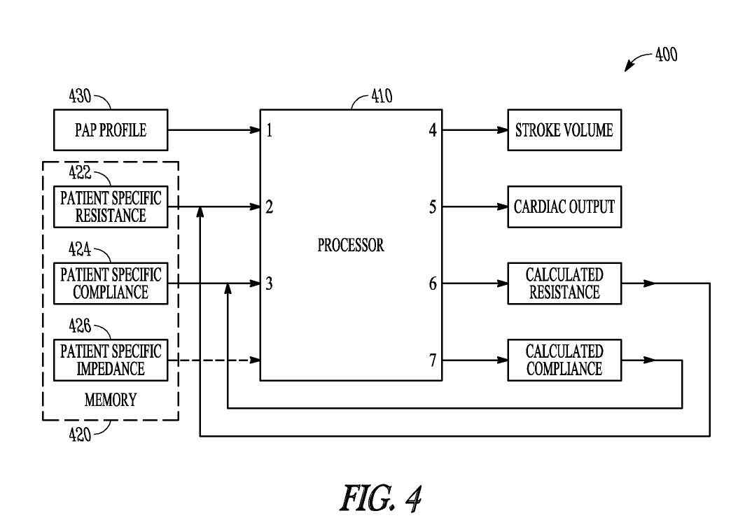

FIG. 3 illustrates an example of a system 300 and portions of an environment in which system 300 operates. In an example, the system 300 includes an implantable pulmonary artery pressure (PAP) sensor 320, an implantable medical device 301, leads 205 and 310, an external system 302, a communication link 324 between the PAP sensor 320 and the implantable medical device 301, a telemetry link 326 between the PAP sensor 320 and the external system 302, and a telemetry link 303 between the implantable medical device 301 and the external system 302. In an example, the system 300 includes an implantable or other cardiac rhythm management (CRM) system.

In FIG. 3, the implantable PAP sensor 320 and the implantable medical device 301 are implanted in a body 390 that has a pulmonary artery 318 connected to a heart 319. The right ventricle of the heart 319 pumps blood through the pulmonary artery 318 to the lungs of body 390 to get the blood oxygenated. The implantable PAP sensor 320 is a pressure sensor configured for being affixed to a portion of the interior wall of the pulmonary artery 318 to sense a PAP signal.

In the example of FIG. 3, the PAP sensor 320 can be delivered, positioned, or anchored in a pulmonary artery 318 of a subject, such as described in the commonly assigned Chavan et al. U.S. patent application Ser. No. 11/216,738 entitled “DEVICES AND METHODS FOR POSITIONING AND ANCHORING IMPLANTABLE SENSOR DEVICES,” (herein “Chavan et al. '738”) which is hereby incorporated by reference in its entirety, including its disclosure of delivering, positioning, and anchoring a physiologic parameter sensor, such as a pressure sensor, to a bodily vessel, such as the pulmonary artery. In other examples, other methods of delivering, positioning, or anchoring physiologic parameter sensors can be used.

The sensed PAP signal is transmitted to the implantable medical device 301 through the communication link 324. In an example, the sensed PAP signal is transmitted to the external system 302. In an example, the communication link 324 includes a wired communication link formed by a lead connected between the implantable PAP sensor 320 and the implantable medical device 301. In another example, the communication link 324 includes an intra-body wireless telemetry link. In a specific example, the intra-body wireless telemetry link is an ultrasonic telemetry link. The implantable medical device 301 can include a sensor signal processing system 321 that can receive and process the PAP signal sensed by the implantable PAP sensor 320. In an example, the implantable medical device 301 comprises a cardiac rhythm management system that is configured to provide one or more of a pacing therapy, a defibrillation therapy, an anti-tachyarrhythmia pacing therapy, a resynchronization therapy, or a neural stimulation therapy. In an example, the implantable medical device 301 further includes one or more of other monitoring and/or therapeutic devices such as a drug or biological material delivery device. The implantable medical device 301 can include a hermetically sealed can housing an electronic circuit, such as to help sense one or more physiological signals or to help deliver one or more therapeutic electrical pulses or other therapies. The hermetically sealed can, in certain examples, can also provide an electrode, such as for electrical sensing or electrical energy delivery.

In some examples, sensor signal processing system 321 can be implemented by a combination of hardware and software. In some examples, the signal processing system 321 can include elements such as those referred to as modules below, which can include an application-specific circuit constructed to perform one or more particular functions or a general-purpose circuit that can be programmed to perform one or more functions. Such a general-purpose circuit can include, but is not limited to, a microprocessor or a portion thereof, a microcontroller or portions thereof, or a programmable logic circuit or a portion thereof.

In an example, the communication link 303 transmits data representative of the PAP signal sensed by implantable PAP sensor 320 such as to be processed or stored in implantable medical device 301. Examples of an implantable PAP sensor and sensor signal processing are described in U.S. patent application Ser. No. 11/249,624, entitled “METHOD AND APPARATUS FOR PULMONARY ARTERY PRESSURE SIGNAL ISOLATION,” filed on Oct. 13, 2005, assigned to Cardiac Pacemakers, Inc., which is incorporated herein by reference in its entirety.

The external system 302 can allow programming of the implantable medical device 301 and can receive information about one or more physiologic or other signals acquired by the implantable medical device 301. In an example, the external system 302 can include a programmer. In another example, the external system 302 can include a patient management system, such as an external device near the implantable medical device 301, a remote device in a relatively distant location from the external device and the implantable medical device 301, and a telecommunication network linking the external device and the remote device. The patient management system can provide access to the implantable medical device 301 from a remote location, such as for monitoring patient status or adjusting one or more therapies. The telemetry link 303 can include a wireless communication link providing bidirectional data transmission between the implantable medical device 301 and the external system 302. In an example, the telemetry link 303 can include an inductive telemetry link. In another example, telemetry link 303 can include a far-field radio-frequency telemetry link. The telemetry link 303 can provide data transmission from the implantable medical device 301 to the external system 302. This can include, for example, transmitting real-time physiological data acquired by the implantable medical device 301, extracting physiological data acquired by and stored in the implantable medical device 301, extracting therapy history data stored in the implantable medical device 301, or extracting data indicating an operational status of the implantable medical device 301 (e.g., battery status or lead impedance). The telemetry link 303 can also provide data transmission from the external system 302 to the implantable medical device 301. This can include, for example, programming the implantable medical device 301 to acquire physiological data, programming the implantable medical device 301 to perform at least one self-diagnostic test (such as for a device operational status), programming the implantable medical device 301 to enable an available monitoring or therapeutic function, or programming the implantable medical device 301 to adjust one or more therapeutic parameters such as pacing or cardioversion/defibrillation parameters.

In an example, processor 321 may be located in the external system 302. In an example, PAP sensor 320 directly communicates to external system 302.

In an example, the PAP sensor 320 can include an implantable pressure sensor placed in the PA to sense the PAP signal, such as that disclosed in the commonly assigned Stahmann U.S. patent application Ser. No. 11/249,624 entitled “METHOD AND APPARATUS FOR PULMONARY ARTERY PRESSURE SIGNAL ISOLATION,” which is hereby incorporated by reference in its entirety, including its disclosure of sensing the PAP signal using the implantable pressure sensor placed in the PA. In other examples, other pressure sensor configurations can be used to sense the PAP signal.

The PAP sensor 320 can be configured to communicate with one or more processors in the sensor signal processing system 321, a cardiac rhythm management device, an external medical device, or a combination or permutation of the one or more than one Implantable Medical Device (IMD), the sensor signal processing system 321, the cardiac rhythm management device, and the external medical device. Certain examples of such sensors, sensor configurations, and communication systems and methods are discussed in more detail in the Mazar et al. U.S. patent application Ser. No. 10/943,626 entitled “SYSTEMS AND METHODS FOR DERIVING RELATIVE PHYSIOLOGIC PARAMETERS;” the Von Arx et al. U.S. patent application Ser. No. 10/943,269 entitled “SYSTEMS AND METHODS FOR DERIVING RELATIVE PHYSIOLOGIC MEASUREMENTS USING AN EXTERNAL COMPUTING DEVICE;” the Von Arx et al. U.S. patent application Ser. No. 10/943,627 entitled “SYSTEMS AND METHODS FOR DERIVING RELATIVE PHYSIOLOGIC PARAMETERS USING A BACKEND COMPUTING SYSTEM;” and the Chavan et al. U.S. patent application Ser. No. 10/943,271 entitled “SYSTEMS AND METHODS FOR DERIVING RELATIVE PHYSIOLOGIC PARAMETERS USING AN IMPLANTED SENSOR DEVICE;” and the U.S. patent application Ser. No. 10/943,271 entitled “SYSTEMS AND METHODS FOR DERIVING RELATIVE PHYSIOLOGIC MEASUREMENTS USING AN IMPLANTED SENSOR DEVICE,” all assigned to Cardiac Pacemakers, Inc., all of which are incorporated herein by reference in their entirety, and which are collectively referred to as the “Physiologic Parameter Sensing Systems and Methods Patents” in this document.

In the example of FIG. 3, the sensor signal processing system 321 can be communicatively coupled to the PAP sensor 320. Generally, the sensor signal processing system 321 can be configured to compute the stroke volume or cardiac output of a patient's heart. This can involve using information from the PAP sensor 320, such as by using at least one detected PA pressure characteristic or other information received from the PAP sensor 320.

In an example, the sensor signal processing system 321 can be configured to detect at least one PA pressure characteristic, such as a PA diastolic pressure (“PAD”), a PA systolic pressure (“PAS”), a mean (or other central tendency) PAP, a PA end-diastolic pressure (“PAEDP”), a rate of pressure change in the PA (“PA dP/dt”), a PA pulse pressure (“PAPP”), or other PA pressure characteristic, using PAP information, such as the PAP signal, from the PA pressure sensor 305.

In an example, the sensor signal processing system 321 can be configured to detect at least one signal correlative to at least one LV pressure characteristic, such as a LV pressure, a LV diastolic pressure, a LV systolic pressure, a LVEDP, a mean (or other central tendency) LV pressure, a LV volume, a LV dP/dt, or other LV pressure characteristic, such as by using PAP information from the PAP sensor 120.

In the example of FIG. 3, the sensor signal processing system 321 can include a time interval detector. In an example, the time interval detector can be configured to detect at least one time interval between at least a first feature of the PAP signal occurring at a first time and at least a second feature of the PAP signal occurring at a second time. In certain examples, the sensor signal processing system 321 can be configured to compute one or more than one output, such as by using at least one mathematical operation and one or more than one interval. Examples can include computing the difference between more than one interval, computing an average (or other central tendency measure) of more than one interval, or computing one or more than one other output using at least one mathematical operation.

In an example, the sensor signal processing system 321 can be configured to provide a notification of the computed cardiac output or estimated stroke volume to an external device 302, such as an external repeater, or other device capable of communicating with the processor 321. In certain examples, the external device, IMD, or other device can be configured to communicate, such as by an e-mail or other communication, to a user, such as a physician or other caregiver, or the subject.

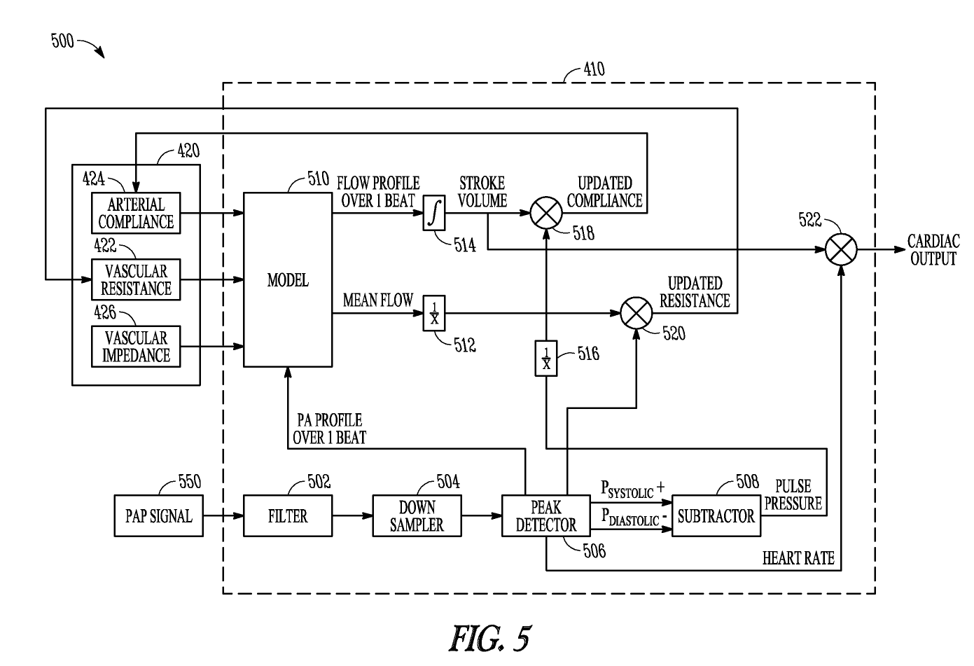

FIG. 4 illustrates generally an example of a processing system 400 configured to receive a pulmonary artery pressure signal, a stored patient-specific lumped parameter that can include vascular resistance and patient-specific arterial compliance. Using this information, the processing system 400 can generate one or more values for stroke volume or cardiac output for a patient 390. In an example, the processing system 400 can include a processor 410 configured to receive a PAP profile 430 at input 1, a patient-specific vascular resistance 422 at input 2, and a patient-specific arterial compliance 424 at input 3. In an example, the patient-specific vascular resistance 422 and patient-specific arterial compliance are stored in a memory 420. In an example, memory 420 includes a lumped parameter related to a patient-specific vascular impedance 426. In an example, the patient-specific vascular impedance 426 is provided to the processor 410 in conjunction with the patient-specific vascular resistance 422 and patient-specific arterial compliance 424.

In an example, the processor 410 can be configured to generate one or more of a stroke volume at output 4, a cardiac output at output 5, a calculated vascular resistance at output 6, or a calculated arterial compliance at output 6. In an example, the calculated vascular resistance and the calculated arterial compliance values are fed back to the inputs 2 and 3 to be used by the processor 410 in a later processing cycle.

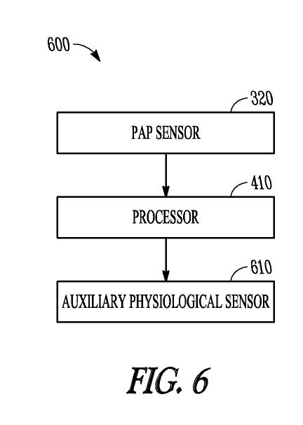

FIG. 5 is an example of a system 500 showing a detailed view of the processing system in FIG. 4 for generating a cardiac output signal representing the cardiac output of the patient 390. In this example, the system 500 can include the memory 420 and the processor 410. In an example, the processor 410 can include a model 510, a filter 502, a down sampler 504, a peak detector 506, a subtracting module 508, inversing modules 512, 516, an integrator 514, and multipliers 518, 520, and 522, which can be coupled as shown in FIG. 5. In an example, the model 510 can be configured to determine blood flow by solving Equation 1, which can be used to represent the 2-element Windkessel model. In an example, the model 510 can be configured to determine blood flow by solving Equation 2, which can be used to represent the 3-element Windkessel model.

In an example, the PAP signal 550 is received at the filter 502. In an example, the filter 502 can include either a Chebyshev filter or a Butterworth filter that can be configured to filter out or attenuate noise in the PAP signal received from the PAP sensor 320. In an example, the filtered PAP signal from the filter 502 can be received by the down-sampler 504, which can reduce the sampling rate of the filtered PAP signal. In an example, the peak detector 506 can provide a pulmonary artery pressure profile over a heart beat to the model 510. In an example, the peak detector 506 can be configured to determine the heart rate of the patient, such as from the PAP signal using a systolic period and a diastolic period. In an example, the systolic period and the diastolic period can be determined by identifying a dicroctic notch within a heart cycle in the PAP signal. In an example, the detected systolic pressure (P_{systlolic}) and diastolic pressure (P_{diastolic}) can be provided to a subtractor module 508, which can calculate the pulse pressure by calculating a difference between P_{systolic} and P_{diastolic}. In an example, the peak detector 506 can calculate the mean (or other central tendency) pressure and can provide it to the multiplier 520, which also receives the inverse of mean blood flow that is generated by the model 510.

In an example, the inverse of pulse pressure can be provided to the multiplier 518 along with the stroke volume received from the integrator 514. The output of the multiplier 518 can include the updated arterial compliance, which can be used to replace initial patient-specific arterial compliance 424. The output of the multiplier 520 can include the updated vascular resistance, which can be used to replace the initial patient-specific vascular resistance 422. Finally, the multiplier 522 can be configured to receive the stroke volume from the integrator 514 and the heart rate from the peak detector 506 to determine the cardiac output for the patient 390.

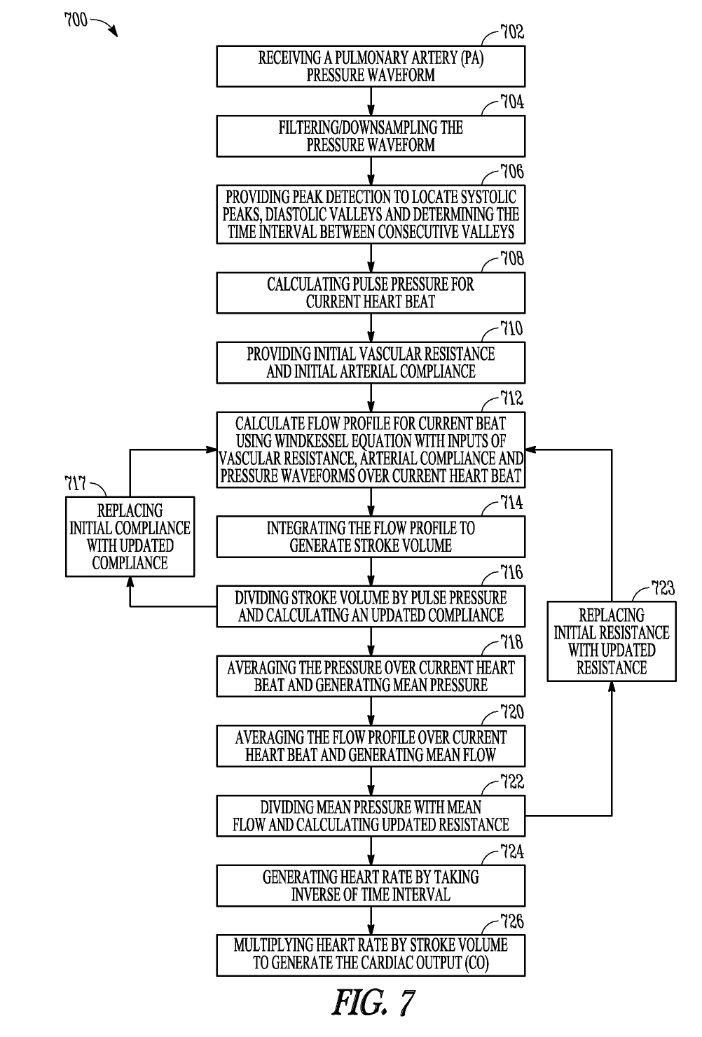

FIG. 6 illustrates generally an example of a system 600 including the PAP sensor 320, the processor 410, and an auxiliary physiological sensor 610. In certain examples, one or more of the PAP sensor 320, the processor 410, or the auxiliary physiological sensor 610, can include an implantable component, an external component, or a combination or permutation of an implantable component and an external component. For example, the processor 410 can be implantable, external, or distributed across both implantable and external locations.

Generally, the auxiliary physiological sensor 610 can be configured to sense a different physiological signal of a subject, such as a physiological signal other than the PAP signal of the subject. The auxiliary physiological sensor 610 can include an implantable or external sensor configured to sense a different physiological signal of the subject, such as a cardiac sensor configured to sense a cardiac signal of the subject, a heart sound sensor configured to sense a heart sound signal of the subject, a right ventricular pressure sensor configured to sense a right ventricular pressure signal of the subject, a left ventricular pressure sensor configured to sense a left ventricular pressure signal of the subject, a blood pressure sensor configured to sense a blood pressure signal of the subject, an oxygen saturation sensor configured to sense an oxygen saturation signal of the subject, an impedance sensor to sense a cardiac impedance of the subject, an accelerometer, such as a lead based accelerometer, configured to sense an acceleration or deceleration of the subject, such as an acceleration or deceleration of the left ventricle of the subject, a physical activity sensor configured to sense a physical activity signal of the subject, a posture sensor configured to sense a posture of a subject, or other auxiliary physiological sensor configured to sense another physiological signal of the subject.

In an example, the processor 410 can be communicatively coupled to the auxiliary physiological sensor 610 and the PAP sensor 320. The processor 410 can be configured to receive information from the auxiliary physiological sensor 610 and to receive information from the PAP sensor 320, such as the PAP signal. In an example, the processor 410 can be configured to compute the stroke volume and cardiac output of the heart using information from the PAP sensor 320 and information from the auxiliary physiological sensor 610.

FIG. 7 illustrates an example of a method 700 to estimate the stroke volume or cardiac output of a patient.

At 702, the method 700 can include receiving the pulmonary artery pressure signal. The PAP signal can include any signal indicative of at least a portion of a PAP of a PA of a subject. In an example, the PAP signal can be sensed using the PA sensor 320.

At 704, the method 700 can include at least one of filtering or down-sampling of the pressure waveform received at 702. In an example, the received PAP signal can be filtered using the filter 502. In an example, the filtered PAP signal can be down-sampled using the down-sampler 504.

At 706, the method 700 can include performing peak detection of the down-sampled waveform, such as to locate one or more systolic peaks or one or more diastolic valleys, and determining the time interval between consecutive valleys. In an example, the peak detection can be performed using the peak detector module 506. In an example, the systolic and the diastolic periods can be identified using a dicroctic notch in a heart cycle of the PAP signal.

At 708, the method 700 can include calculating the pulse pressure for a current heart beat. In an example, the pulse pressure can be calculated by forming a difference between the systolic pressure (P_{systolic}) and the diastolic pressure (P_{diastolic}) at the subtractor module 508.

At 710, the method 700 can include providing an initial patient specific vascular resistance model parameter 422 and an initial patient-specific arterial compliance model parameter 424 to the model 510. In an example, the initial patient-specific arterial compliance model parameter 422, the initial patient-specific vascular resistance model parameter 424 and the initial patient-specific vascular impedance model parameter 426 can be stored in the memory 420. In an example, the initial patient-specific vascular resistance model parameter and the initial patient-specific arterial compliance model parameter can be derived using at least one physiological characteristic of the patient. An example of a physiological characteristic that can be used to determine the above model parameters includes the physical dimensions of the patient's heart, which can be determined using a either CT-scan or ultrasound imaging.

At 712, the method 700 can include calculating the flow profile for the current beat at the model 510 using any one of the two Windkessel equations (using equation 1 in the case of a two-element model or equation 2 in the case of a three-element model).

At 714, the method 700 can include integrating the flow profile over a heart beat to determine a stroke volume. In an example, the integrator 514 can perform the integration on the received output flow profile from the model 510.

At 716, the method 700 can include dividing the stroke volume by the pulse pressure and calculating an updated arterial compliance.

At 717, the method 700 includes replacing in memory 420 the initial arterial compliance 422 with the updated arterial compliance calculated at block 716.

At 718, the method 700 can include averaging the pressure over the current heart beat and determining a mean pressure.

At 720, the method 700 includes averaging the flow profile over current heart beat and determining a mean blood flow.

At 722, the method 700 can include dividing the mean pressure by the mean blood flow and calculating an updated vascular resistance. In an example, the module 512 provides the inverse of mean blood flow to the multiplier 520, which outputs the updated vascular resistance.

At 723, the method 700 can include replacing in the memory 420 the initial vascular resistance 424 with the updated vascular resistance calculated at 722.

At 724, the method 700 can include generating heart rate by taking the inverse of time interval between contractions. In an example, the heart rate is provided to the multiplier 522 by the peak detector module 508.

At 726, the method 700 can include multiplying the heart rate received from the peak detector module 506 with the stroke volume received from the integrator module 514 to generate the cardiac output. In an example, the heart rate and stroke volume can be multiplied using the multiplier 522.

### Additional Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown and described. However, the present inventors also contemplate examples in which only those elements shown and described are provided.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, the code may be tangibly stored on one or more volatile or non-volatile computer-readable media during execution or at other times. These computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. An apparatus comprising:
an input receiving a pressure sensor signal from an implantable pressure sensor;
an output providing an output signal used to calculate a cardiac output;
a memory configured to store a patient-specific vascular resistance model parameter and a patient-specific arterial compliance model parameter; and
a processor coupled to the input, the output and the memory, wherein the processor is configured to:
receive the pressure sensor signal, the pressure sensor signal including a systolic period and a diastolic period;
determine a heart rate (HR) of a heart cycle;
provide an iteratively-updating model that relates the pressure sensor signal and HR to at least one of a stroke volume (SV) and a cardiac output (CO), and that extracts a measure of a pulse pressure derived from the pressure sensor signal, the model using a patient-specific vascular resistance model parameter and a patient-specific arterial compliance model parameter;
calculate the CO using the HR, the pressure sensor signal, and the model;
iteratively update the patient-specific vascular resistance model parameter using the output signal from the model; and
iteratively update the patient-specific arterial compliance model parameter using the output signal from the model.

2. The apparatus of claim 1, wherein the implantable pressure sensor includes a pulmonary artery pressure (PAP) sensor disposed in a pulmonary artery of a patient and configured to generate the pressure sensor signal.

3. The apparatus of claim 1, wherein the processor receives a patient-specific vascular impedance model parameter stored in the memory.

4. The apparatus of claim 1, wherein the processor is configured to determine a pulmonary blood flow profile over the heart cycle using the pressure sensor signal, the vascular resistance model parameter and the arterial compliance model parameter that are stored in the memory.

5. The apparatus of claim 1, wherein the processor is configured to determine the stroke volume by integrating the pulmonary blood flow profile over the heart cycle.

6. The apparatus of claim 1, wherein the processor is configured to determine a second arterial compliance model parameter using the pressure sensor signal.

7. The apparatus of claim 6, wherein the processor is configured to determine the second arterial compliance model parameter using the stroke volume and a pulse pressure extracted from the pressure sensor signal.

8. The apparatus of claim 4, wherein the processor is configured to determine a second vascular resistance model parameter using the pressure sensor signal.

9. The apparatus of claim 8, wherein the processor is configured to determine a second vascular resistance model parameter using a central tendency of the pressure sensor signal and a central tendency of the pulmonary blood flow profile over the heart cycle.

10. The apparatus of claim 8, wherein the processor is configured to replace the vascular resistance model parameter with the second vascular resistance model parameter.

11. The apparatus of claim 6, wherein the processor is configured to replace the arterial compliance model parameter with the second arterial compliance model parameter.

12. The apparatus of claim 2 configured to filter and down-sample the pressure sensor signal and generate a pulmonary blood flow profile.

13. The apparatus of claim 1, wherein the processor is configured to determine the heart rate and the heart cycle by identifying the systolic period and the diastolic period in the pressure sensor signal.

14. The apparatus of claim 13, wherein the processor is configured to identify the systolic period and the diastolic period by identifying a dicroctic notch in the pressure sensor signal during the heart cycle.

15. The apparatus of claim 14, wherein the processor identifies the dicrotic notch using peak detection.

16. The apparatus of claim 14, wherein the processor identifies the dicrotic notch using a physiological signal generated from a second physiological sensor.

17. The apparatus of claim 16, wherein the second physiological sensor includes at least one of a heart sound sensor or an ECG monitor.

18. The apparatus of claim 1, wherein the processor is configured to determine a central tendency of the CO over a specified number of heart cycles.

19. The apparatus of claim 1, comprising a posture sensor as an input to normalize cardiac output calculations.

20. The apparatus of claim 1, comprising attenuating a respiration effect of the patient from the calculated CO.

21. The apparatus of claim 1, wherein the processor is disposed in an implanted device capable of being communicatively coupled to the implantable pressure sensor

22. The apparatus of claim 21, wherein the implantable medical device is configured to communicate with an external device.

23. The apparatus of claim 1, wherein the processor is disposed in an external device capable of being communicatively coupled to the implantable pressure sensor.

24. A system comprising:
means for receiving a pulmonary artery pressure (PAP) signal from a pulmonary artery pressure sensor disposed in a pulmonary artery of a patient, the PAP signal including a systolic period and a diastolic period;
means for determining a heart rate (HR) of a heat cycle;
means for providing an iteratively-updating model that relates PAP and HR to at least one of a stroke volume (SV) and a cardiac output (CO), and that extracts a measure of central tendency of pulse pressure (MPP) from the PAP, the model using a patient-specific vascular resistance model parameter and a patient-specific arterial compliance model parameter;
means for calculating the CO using the HR, the PAP signal, and the model;
means for iteratively updating the patient-specific vascular resistance model parameter using the model outputs; and
means for iteratively updating the patient-specific arterial compliance model parameter using the model outputs.

25. The system of claim 24, wherein the means for receiving a pulmonary artery pressure includes an external device.
